(19) United States
(12) Patent Application Publication
(10) Pub. No.: US 2001/0037076 A1
(43) Pub. Date: Nov. 1, 2001

(54) Temperature and compression treatment garment for the chest

(76) Inventor: Sharon Rene?apos;e Shelton, Huntsville, AL (US)

Correspondence Address: LANIER FORD SHAVER & PAYNE, P O BOX 2087, HUNTSVILLE, AL, 35804, US

(21) Appl. No.: 09/803,811

(22) Filed: Mar. 12, 2001

Related U.S. Application Data
(60) Provisional application No. 60/188,949, filed on Mar. 13, 2000.

(51) Int. Cl. A61F 5/00

(52) U.S. Cl. 602/7; 602/5

(57) ABSTRACT

A temperature compression treatment garment for providing therapy to the chest and/or breasts for relief of pain and discomfort, comprising a band which is wrapped around the upper torso at the level of the chest and/or breasts and which applies compression variably and independently horizontally across the chest. The band has front and back shoulder straps which connect to apply compression variably and independently vertically across the chest. The band also has a frontal flap which forms a pocket for the insertion of a thermal device, such as a gel-pack, to heat or cool the chest and/or breasts of a user.

CROSS REFERENCE TO RELATED APPLICATIONS

This application claims the benefit of an earlier filed co-pending U.S. Provisional Patent Application Serial No. 60/188,949, filed Mar. 13, 2000, the disclosure of which is hereby incorporated herein by reference.

### BACKGROUND OF THE INVENTION

1. Field of the Invention

This invention relates to garments for the chest and/or breasts, and in particular, to garments for therapeutic compression and temperature treatment for the chest and/or breasts.

2. Background of the Invention

There are numerous medical procedures or medical conditions which result in pain and discomfort to the chest and/or breast regions of a patient. Various chemical agents such as narcotic analgesic and anti-inflammatory agents may be used to relieve the pain and discomfort but these therapeutic agents often have undesirable side effects, may not be tolerated by the patient, or may have inadequate efficiency.

Some of the medical conditions which lead to pain and discomfort in the chest and/or breast region include, for example, lateral or bilateral mastectomy, breast augmentation or reduction, open heart surgery, coronary bypass, and certain traction procedures. Other conditions also result in residual pain such as postpartum breast swelling, premenstrual syndrome, breast or nipple infection, or traumatic injury.

It is known in the art that compression of the breast or chest area will help relieve pain and discomfort associated with medical procedures, disease conditions, or traumatic injury. it is also known that applied heat or cold (thermal treatment) will produce relief Furthermore, the combination of both compression and thermal treatment may produce superior relief, and this combination may be the most desirable treatment. However, the application of both compression and temperature therapy itself can produce discomfort to the patient if not applied appropriately. For example, present methods and devices for combination therapy apply pressure to the breasts or chest and also apply pressure to the temperature regulating devices as well. Compressing the temperature regulating devices against the chest or breasts can be very discomforting to the patient, even to the point where the treatment is not useful.

The present invention overcomes these problems and limitations by providing a treatment garment which applies compression and thermal treatment variably and independently to the chest and/or breasts. In addition, the present invention provides for compression both horizontally and vertically across the chest and/or breasts.

### SUMMARY OF THE INVENTION

The garment of the present invention has a band that wraps around the upper body in an annular fashion at the level of the chest, under the arms. The band has a first end having a hook or pile arrangement and an opposite second end having a hook or pile arrangement. The band can be reversibly closed around the chest with any desired degree of compression, producing compression horizontally across the chest and/or breasts, from the bottom to the top of the chests and or breasts, using the hook and pile arrangements on the first and second ends of the band. The upper edge of the band has two pairs of shoulder straps, the first pair at the first end of the band (back of the garment), each strap having hook or pile arrangements, and the second pair at the second end of the band (front of the garment), each having hook or pile arrangements. The first pair of shoulder straps are placed over the shoulders to connect reversibly with the second pair of shoulder straps, forming a bra-like structure when the two ends of the band are connected. The shoulder straps can be reversibly connected over the shoulders with any desired degree of compression producing compression vertically across the chest and/or breasts, from one side to the other, using the hook and pile arrangements on the shoulder straps. On the bottom edge of the band at the second end is a frontal flap having hook or pile arrangements which connect with the hook or pile arrangements on the second pair of shoulder straps and/or on the upper edge at the second end of the band to form a pocket which can contain a thermal device such as, for example, a gel-pack, which can heat or cool the chest and/or breasts. In this manner, the garment of the present invention can apply variable compression to the chest and/or breasts horizontally and/or vertically, and can, independently of the compression, also, apply heat or cold to the chest and/or breasts.

An advantage of the present invention is that the garment can be constructed of a single piece of material.

Another advantage of the present invention is that horizontal and vertical compression can be applied to the chest and/or breasts variably, independently, and simultaneously.

Another advantage of the present invention is that thermal treatment may be applied to the chest and/or breasts independent of the degree or nature of compression produced by the annular band.

Another advantage of the present invention is that compression and thermal treatment may be used simultaneously or independently to relieve pain and/or discomfort of the chest and/or breasts.

### BRIEF DESCRIPTION OF THE DRAWINGS

FIG. 1 shows the outside surface of the garment of the present invention in an open flat position.

FIG. 2 shows the inside surface of the garment of the present invention in an open flat position.

FIG. 3 shows the garment of the present invention in an annular open position with the shoulder straps open and extending upward, and a frontal flap extending downward.

FIG. 4 shows the garment of the present invention in an annular closed position with the shoulder straps open and extending upward and the frontal flap extending downward.

FIG. 5 shows the garment of the present invention in an annular closed position with the shoulder straps closed and the frontal flap extending downward.

FIG. 6 shows the garment of the present invention in an annular closed position with the shoulder straps closed and the frontal flap extending upward and attached to the shoulder straps.

### DETAILED DESCRIPTION OF THE PREFERRED EMBODIMENTS

While the following description details the preferred embodiments of the present invention, it is to be understood that the invention is not limited in its application to the details of construction and arrangement of parts illustrated in the accompanying drawings, since the invention is capable of other embodiments and of being practiced in various ways.

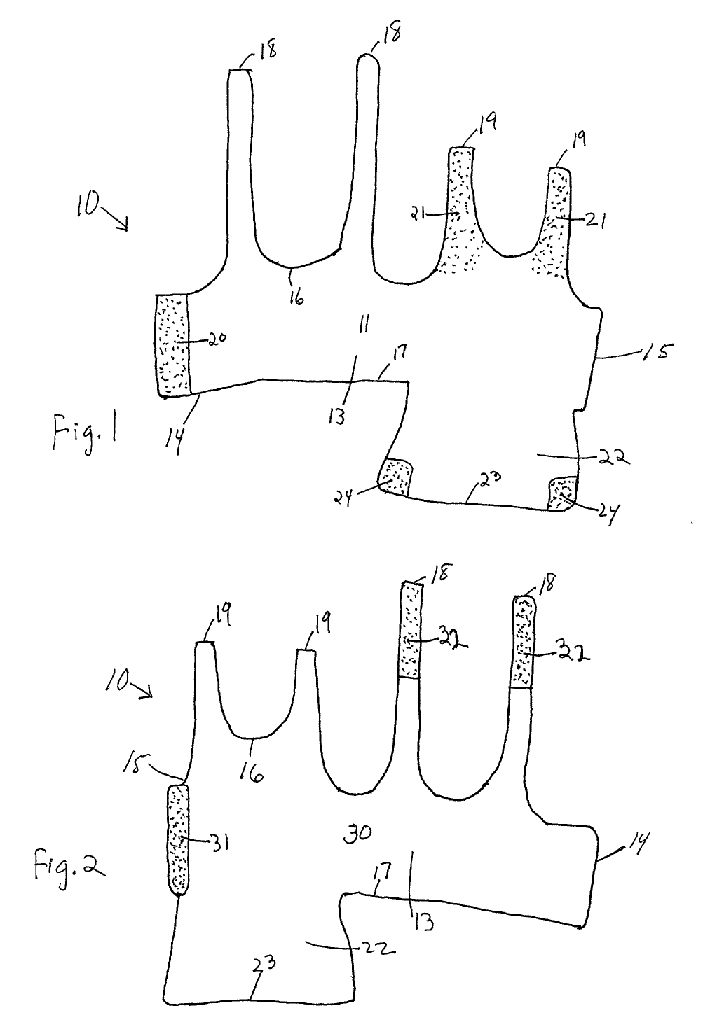

FIG. 1 shows the outside surface 11 of garment 10 of the present invention in an open flat position. The garment has a band 13 having a first end 14 (back of garment) and a second opposite end 15 (front of garment), an upper edge 16 and a bottom edge 17. Upper edge 16 has a pair of shoulder straps 18 oriented towards first end 14 (back shoulder straps) and a pair of shoulder straps 19, oriented towards second end 15 (front shoulder straps). First end 14 has a hook or pile arrangement 20 on the outside surface. Shoulder straps 19 have hook or pile arrangements 21 on the outside surface. Bottom edge 17 has a frontal flap 22 attached at second end 15, with an external edge 23 extending away from bottom edge 17. External edge 23 of frontal flap 22 has hook or pile arrangements 24.

FIG. 2 shows the inside surface 30 of garment 10 of the present invention in an open flat position, and several of the features shown in FIG. 1, plus the hook or pile arrangement 31 on the inside surface of second end 15 and the hook or pile arrangements 32 on the inside surfaces of shoulder straps 18.

FIG. 3 shows the garment 10 of the present invention in an annular position with ends 14 and 15 in an open position. Shoulder straps 18 having hook or pile arrangements 32 are shown extending upward and frontal flap 22 is shown extending downward.

FIG. 4 shows the garment 10 of the present invention in an annular closed position with second end 15 folded over first end 14, and hook or pile arrangement 31 on second end 15 reversibly locking with hook or pile arrangement 20 on first end 14, maintaining band 13 in an annular closed position.

FIG. 5 further shows shoulder straps 18 folded over shoulder straps 19, and hook or pile arrangement 32 on shoulder strap 18 reversibly locking with hook or pile arrangement 21 on shoulder strap 19.

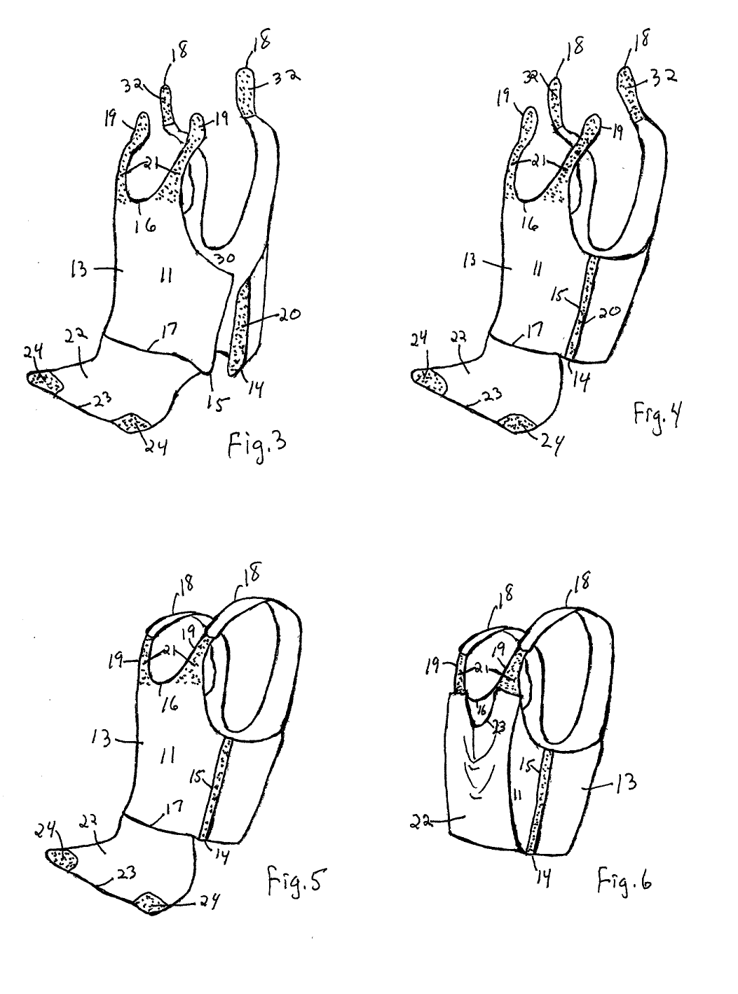

FIG. 6 further shows frontal flap 22 extending upward and reversibly locking with shoulder straps 19, by hook or pile arrangements 24 on external end 23 reversibly locking with hook or pile arrangements 21 on shoulder straps 19. When frontal flap 22 is thus reversibly attached to straps 19 a pocket is formed by frontal flap 22 and outside surface 11 of band 13 over the chest and/or breasts. A thermal device may be placed in this pocket to heat or cool the chest and/or breasts. Frontal flap 22 may be opened or closed without altering the closure of shoulder straps 18 and 19 or first and second ends 14 and 15 of band 13.

As depicted in the figures above, second end 15, having shoulder straps 19, forms the front of garment 10. First end 14, having shoulder straps 18, forms the back of garment 10. Band 13, from bottom edge 17 to upper edge 16, extends from the bottom portion to the top portion of the chest and/or breasts. Although second end 15 fits over first end 14, first end 14 could fit over second end 15, with appropriate changes in the placements of hook or pile arrangements 20 and 31. Likewise, although shoulder straps 18 fit over shoulder straps 19, shoulder straps 19 could fit over shoulder straps 18, with appropriate changes in the placements of the hook and pile arrangements 32 and 21. Although frontal flap 22 reversibly attaches to shoulder straps 19 in FIG. 6, frontal flap 22 could also attach to upper edge 16 of band 13 or to both shoulder straps 19 and upper edge 16.

The hook or pile arrangements 20, 21, 24, 31, and 32 shown in the Figures may have either a hook fastener or a pile fastener, but the element to which each locks will have the opposite fastener. For example, if first end 14 has a hook arrangement 20, then second end 15 will have a pile arrangement 31, and vice versa. The hook and pile arrangement is similar to that provided under the registered trademark Velcro®. Although hook and pile arrangements are preferred, other reversible closing or locking mechanisms may be used, such as, for example, snaps, belts, ties, and the like.

The garment of the present invention may be constructed in one piece as shown in FIGS. 1 and 2, and folded and locked to form a bra-like structure fitting around the upper torso under the arms, thereby covering the chest and/or breasts. the band 13 of the garment of the present invention may be constructed to be closed and locked on either the right side of the chest or on the left side by changing the orientation of the inside and the outside of the garment, with corresponding changes in the orientation of the hook and pile arrangements, as desired.

The garment of the present invention may be constructed of any suitable cloth and/or plastic material. The preferred material will be soft, stretchable (in two directions), resilient, and elastic, and be porous or permeable sufficiently to allow moisture to evaporate and escape from the portion of the body covered by the garment, and to conduct heat. Several such materials are known in the art and include, for example, those made of polyamide, polypropylene, and/or polyester fabrics, preferably polypropylene fabric sold under the trademark Silkylene™.

The garment of the present invention can be used to apply variable compression to the chest and/or breasts in horizontal and/or vertical directions while, simultaneously and independently, applying heat or cold to the chest and/or breasts. The application of compression and heat or cold to the chest and/or breasts is useful in relieving pain and discomfort in a variety of conditions and disorders, including, for example, lateral or bilateral mastectomy, breast augmentation or reduction, open heart surgery, coronary bypass, certain traction procedures, postpartum breast swelling, premenstrual syndrome, breast or nipple infection, and any like conditions which produce trauma or induce pain in the chest and/or breasts.

In use, band 13 of the garment is wrapped around the upper torso in an annular fashion at the chest and/or breast level, under the arms. One end of band 13 is reversibly attached to the opposite end of band 13 applying any degree of compression desired prior to attaching and reversibly locking one end of the band to the opposite end of the band. Compression is, thus, applied horizontally across the chest and/or breasts from the bottom portion to the top portion of the chest and/or breasts.

The shoulder straps on the back of the garment are then pulled over the shoulders towards the front and reversibly connected to the shoulder straps on the front of the garment. By pulling the back and front shoulder straps towards each other any degree of compression can be created prior to attaching and reversibly locking the back and front shoulder straps together. Compression is, thus, applied vertically across the chest and/or breasts from one side of the chest and/or breasts to the other.

The horizontal and vertical compressions can be adjusted variably and independently. Once the desired compression is created, a thermal device can be placed against the chest and/or breasts to provide heat or cold treatment and be held in place in the pocket created by reversibly attaching the frontal flap to the front shoulder straps and/or upper front edge of the band. The thermal device can be applied or removed independently of the application of compression and without altering or interfering with the compression generated by the band or shoulder straps.

Any thermal device that is adapted to fit over the chest and/or breasts can be used in the present invention. The preferred thermal unit is a gel-pack well-known in the art for applying heat or cold to the body surface. Gel or reusable fluid materials employable in the gel-packs (pouches) of the present invention for heat exchange are well known in the art. For example, one reusable, heatable/chillable gel employable in the pouches could be composed of approximately 40% glycerine, 52% distilled water, and 8% starch. It is further well known in the art to provide plastic film pouches, packages, and containers, and heating or refrigerating package constructions of plastic film material. Typical materials making up such films include polyethylene, cellophane, polypropylene, polyester, etc., and laminations thereof. The preferred gel-pack is reusable, can be heated in any suitable oven or heating device, including, preferably, a microwave oven, and can be placed in a freezer to cool the gel-pack to 0° C. or lower without causing the gel to solidify.

The garment 10 of the present invention may be further adapted so that a thermal device, such as, for example, a gel-pack may be reversibly attached directly to outside surface 11 at opposite end 15 (front of garment 10) directly over the chest, without the use of frontal flap 22 for holding the gel-pack against the chest. The front of garment 10 may have a hook or pile arrangement and the gel-pack may have a corresponding reciprocal hook or pile arrangement so that the gel-pack will adhere to outside surface 11 over the chest when placed against the chest. Although hook and pile arrangements are preferred, other reversible closing or locking mechanisms maybe used, such as, for example, snaps, belts, ties, and the like.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. For example, the position or orientation and surface area of the hook or pile arrangements can be varied as desired. Several pieces of material may be sewn together to make a single garment. Different types of fabrics may be used in a single garment. The garment may have one or more openings for drainage tubes and the like, or connectors, clips, fasteners, and the like to hold or support other objects as needed.

It will be understood that various changes in the details, materials, and arrangements of the parts which have been described and illustrated above in order to explain the nature of this invention may be made by those skilled in the art without departing from the principle and scope of the invention as recited in the following claims.

I claim:

1. A temperature compression treatment garment for the chest, comprising:
a) a band having a front, a back, an upper edge, a bottom edge, a first end, and an opposite second end, said first and second ends being reversibly connectable;
b) a first pair of shoulder straps on said upper edge and said back of said band, and a second pair of shoulder straps on said upper edge and said front of said band, said first pair and said second pair of shoulder straps being reversibly connectable;
c) said band constructed to provide variable horizontal compression and said shoulder straps constructed to provide variable vertical compression across the chest and/or breasts of a user; and
d) a frontal flap on said bottom edge and said front of said band, said frontal flap being reversibly connectable to said second pair of said shoulder straps and/or said upper edge of said band, thereby forming a pocket to hold a thermal device for warming or cooling the chest and/or breasts of a user.

2. The temperature compression treatment garment according to claim 1 wherein said first and second ends of said band, said first pair and said second pairs of shoulder straps, said frontal flap and said upper edge of said band are reversibly connectable with a hook and pile arrangement.

3. The temperature compression treatment garment according to claim 1 wherein the garment is constructed in one piece.

4. The temperature compression treatment garment according to claim 1 wherein the thermal device is a gel-pack.

5. The temperature compression treatment garment according to claim 1 wherein the garment is constructed of cloth and/or plastic and is stretchable in two directions.

6. The temperature compression treatment garment according to claim 5 wherein the plastic is polypropylene.

7. The temperature compression treatment garment according to claim 1 wherein the garment is constructed with openings to accommodate drainage tubes.

8. The temperature compression treatment garment according to claim 4 wherein said gel-pack is reversibly connected directly to said front of said band, without the use of said frontal flap.

9. A temperature compression treatment garment for the chest, comprising:
a) a band having an upper edge and a bottom edge, a first end defining the back of the garment, and an opposite second end defining the front of the garment;
b) connecting means on said first end and said second end so that said first end can be reversibly connected to said second end to maintain said band wrapped around the upper torso of a user in an annular fashion, under the arms at the level of the chest;
c) two pairs of shoulder straps on said upper edge of said band, a first pair of said straps oriented towards said first end and a second pair of said straps oriented towards said second end;
d) a frontal flap oriented towards said second end, attached at said bottom edge of said band;
e) said shoulder straps having connecting means so that said first pair of shoulder straps can extend from the back of a user, over the shoulders of a user, and reversibly connect with said second pair of straps;
f) said connecting means on said band being adjustable to produce variable compression horizontally across the chest and/or breasts of a user;
g) said connecting means on said shoulder straps being adjustable to produce variable compression vertically across the chest and/or breasts of a user; and
h) said frontal flap having connecting means to reversibly connect with said second pair of shoulder straps and/or said upper edge of said band, thereby forming a pocket to hold a thermal device adjacent to the chest and/or breasts of a user.

10. The temperature compression treatment garment according to claim 9 wherein said connecting means are hook and pile arrangements.

11. The temperature compression treatment garment according to claim 9 wherein the garment is constructed in one piece.

12. The temperature compression treatment garment according to claim 9 wherein the thermal device is a gel-pack.

13. The temperature compression treatment garment according to claim 9 wherein the garment is constructed of cloth or plastic and is stretchable in two directions.

14. The temperature compression treatment garment according to claim 13 wherein the plastic is polypropylene.

15. The temperature compression treatment garment according to claim 9 wherein the garment is constructed with openings to accommodate drainage tubes.

16. The temperature compression treatment garment according to claim 12 wherein said gel-pack is reversibly connected directly to said second end, without the use of said frontal flap.

17. A method for providing temperature and compression therapy to the chest for relief of pain and discomfort, comprising:
a) providing a temperature compression treatment garment comprising a band having a front, a back a first end, an opposite second end, an upper edge, a bottom edge, a first pair of shoulder straps on said upper edge and back, a second pair of shoulder straps on said upper edge and front, and a frontal flap on said bottom edge and front;
b) wrapping said band around the upper torso of a user in an annular fashion under the arms at the level of the chest and/or breasts;
c) reversibly connecting said first end of said band to said second end of said band to produce any degree of compression desired horizontally across the chest and/or breasts from the bottom to the top of chest and/or breasts;
d) reversibly connecting said first pair of shoulder straps to said second pair of shoulder straps over the shoulders of a user to produce any degree of compression desired vertically across the chest and/or breast from one side of the chest and/or breasts to the other;
e) reversibly connecting said frontal flap to said upper edge and front of said band and/or to said second pair of shoulder straps to form a pocket; and
f) insertion of a thermal device into said pocket to provide heat or cold therapy to the chest and/or breasts of a user.

18. A method according to claim 17 wherein reversibly connecting further comprises the use of hook and pile arrangements.

19. A method according to claim 17 wherein said band, shoulder straps, and frontal flap are constructed in one piece.

20. A method according to claim 17 wherein said thermal device is a gel-pack.

21. A method according to claim 17 wherein the garment is constructed of cloth or plastic and is stretchable in two directions.

22. A method according to claim 21 wherein the plastic is polypropylene.

23. A method according to claim 17 wherein the garment is constructed with openings to accommodate drainage tubes.

24. A method according to claim 17 wherein said horizontal compression, said vertical compression, or said heat or cold therapy can each be produced independently of the others.

25. A method according to claim 20 wherein said gel-pack is reversibly connected directly to said front of said band, without the use of said frontal flap.
